(19) United States
(12) Patent Application Publication
(10) Pub. No.: US 2012/0207854 A1
(43) Pub. Date: Aug. 16, 2012

(54) METAL POLYSULFIDE-COMPLEX WITH BIOLOGICAL AND CHEMICAL ACTIVITY

(76) Inventors: Ricardo Carrizo-Manzanero, Maracaibo (VE); Rodolfo Salas-Auvert, Maracaibo (VE); Humberto Herrera-Merchan, Maracaibo (VE)

(21) Appl. No.: 13/025,535

(22) Filed: Feb. 11, 2011

(51) Int. Cl. A61K 33/26; A61P 31/12; A61P 31/10; A61P 31/04; A61P 33/00; C25B 9/06; A61P 17/00; A61P 17/02; A61P 1/02; A61P 17/10; A61P 29/00; A61K 33/24; A61P 31/22

(52) U.S. Cl. 424/646; 424/655; 204/242

(57) ABSTRACT

Aqueous solution, cream, hydrogel or spray composition containing compound Sn-On-Xn; where S(n=5-8), O (n=3-7) and X═Fe, Cr or Ni, and most effectively, S7-O4-Fe4 as active ingredient for cutaneous, intradermal and subcutaneous treatment of dermo-epithelial lesions, comprising but not limited to, certain types of skin cancer (basal cell, keratocanthoma and spinocellular carcinomas), conjunctivitis, folliculitis, acne, gingivitis, stomatitis, fungal, dermatomycosis, parasitosis, condylomatosis, balanitis, vaginitis, vaginosis, bacteriosis and endometriosis; by reducing the number of infecting microorganism, not limited to Candida sp., Actinobacillus actinomycetemcomitans, Pseudomonas sp., Porphyromonas gingivalis., Escherichia, sp., Staphyloccocus sp. Streptococcus sp., Herpes simplex I, Herpes simplex II, Coxackievirus A and B, Echovirus, Varicella Zoster (HHV-3), Human Papilloma Virus Gardnerella Vaginalis, Trichomonas vaginalis, Corynebacterium sp., present on the body skin and mucosas. Treating condition may include other abnormal conditions of the skin and epithelium, including insect bites, leukoplakia, oral (aphthous) ulcers and burns, trauma, aphthous, herpervirus infections, stomatitis, herpangina (mouth blisters), a painful mouth infection caused by coxsackieviruses.

### II. BACKGROUND OF THE INVENTION

1. Field of the Invention

The present invention pertains to a method and composition for prevention and treatment of abnormal conditions of the skin and epithelium of bodily surface and orifices.

More particularly, the present invention relates to the use of compound S7-O4-Fe4 which comprises various mechanisms of action but not limited to anti-oxidation, apoptosis modulator, protein charge modifier, selective cell membrane ion channel modulator which may affect disease-infectious agent survival, absorption and function theretofore contributing to growth control and elimination of bacteria, parasites, fungal and viral agents.

2. Description of the Related Art

The use of electrolytic cells is a very old concept as well as the galvanic cell. Both phenomena are used here, which means that an electrolytic cell yielding the compound with electrical energy and the chemical energy of the final compound solution can be illustrated or viewed using a galvanic cell that converts chemical energy into electrical energy and thus supplied by the system itself. Elements of which are from a very old concept going back to the 1800's.

Sulfur-metal complexes are interesting molecules given their chemistry and useful biological activity, including antitumor, antibacterial, antiviral and antiparasitic action. (J. Q. Qu, G. C. Sun, L. F. Wang and L. Qu, Synthesis, characterization, and biological activities of some transition metal(II) complexes with the thiosemicarbazone derived from 4-[1-(4-methylphenylsulfonyl)-1H-indol-3-yl]but-3-en-2-one. Solvak Acad. Sci. Volume 60, Number 3/June, 2006).

Patent application FR 2740039 describes the use of mixture containing aldehydes and bifunctional compounds, including glutaraldehyde, for inhibiting the attachment of pathogenic strains such as Staphylococcus aureus to the keratinocytes and corneocytes, where the last compound may have toxic implications.

Also, hexachlorophene and derivatives are reported as antibacterial substances and used against Propionibacterium acnes and other bacteria. These treatments are in general expensive and harmful both to health and environment.

Alternative, nontoxic treatments include antimicrobial compositions, comprising about 0.01-4.0% by weight of one or more natural selected oils, including rosemary oil, tea tree oil, spearmint oil, peppermint oil, clove oil, lemongrass oil, cedar oil, and cinnamon oil and about 0.1-4.0% by weight of one or more selected organic acids comprising carboxylic acids, ascorbic acid, glutamic acid, fumaric acid, oxalic acid and malonic acid, which exhibit antifungal, bactericidal or bacteriostatic properties on fomites only (USPTO Appl. No. 20090087502).

Application EP-99204489 describes the use of strains of lactobacillus, micrococcus or bifidobacterium selected for their skin cells adhesion capacity and their competitive inhibition properties as probiotics with skin pathogen adhesion to prepare cosmetic compositions capable of stabilizing and regulating the skin pathogenic flora.

Application PCT/EP96/00441 describes the use of sugars and their derivatives as antiadhesion active agents for the treatment of yeast and dermatophyte mycoses.

Organic sulfur containing compounds and their salts are reported for pharmaceutical activity and application as antifungal agents and as antiproliferative agents against mammalian cells, in particular cancer cells and most particularly leukemia-derived cells (United States Patent Application 20030220524).

Several products (lactobacillus acidophilus, deodorized garlic, beta 1,3 D-glucan, neem bark extract, methronidazole, chlorine dioxide, are used to treat bacterial vaginosis and odor discomfort generated by vaginal infection mostly by gardnerella vaginalis and mycoplasma hominis due to an imbalance in the bacteria normally found in the vagina, specifically the lack of normally occurring lactobacilli. (Delia A, Morgante G, Rago G, et al. Minerva Ginecol 2006 June; 58(3):227-31, Ackland, Marie Destinol Vs Femanol For the Treatment of Bacterial Vaginosis. 2 Feb. 2009, M. Simbara, Z. Azarbadb, F. Mojabc and H. Alavi Majdd Phytomedicine 15(12): 1025-1031, 2008, Pages, U.S. Pat. No. 5,935,592—Method for treating vaginitis and endometriosis).

Sulfur containing compounds are reported for treatment of bacterial infections such as macrolide antibiotics, aminoglycosides, lincosamides, oxazolidinones, streptoramins, tetracycline and/or other compounds which bind to bacterial ribosomal RNA and/or to one or more proteins involved in ribosomal protein synthesis in the bacterium. The compounds adversely affect protein expression and have an antibacterial effect. The multibinding compounds of this invention containing from 2 to 10 ligands covalently attached to one or more linkers. Each ligand is macrolide antibiotic, aminoglycoside, lincosamide, oxazolidinone, streptogramin, tetracycline or other compound which binds to bacterial ribosomal RNA and/or one or more proteins involved in ribosomal protein synthesis in the bacterium. (U.S. Pat. No. 6,900,183—Macrolide antibiotics.)

The search for novel, safe and effective clinical therapeutics for cancer treatment is a priority, where most available compounds are non specific and toxic. Recent advances include novel sulfur containing organic antineoplasic molecules (Cardenas, M. E., Sanfridson, A., Cutler, N. S., and Heitman, J. (1998); Natural products that modify signal-transduction cascades for therapeutic intervention (Marks, P. A., Richon, V. M. and Rifkind, R. A. Trends Biotechnol 16:427-33, 2000) Histone deacetylase inhibitors as inducers of differentiation or apoptosis of transformed cells (Nishimune Y, Okabe M. Calmegin J Natl Cancer Inst 92:1210-6, 2005); Taxanes (taxol), derived from the bark of the yew tree, emerged as effective anti-tumour agents on several malignancies (Vaishampayan, U., Parchment, R. E., Jasti, B. R., and Hussain, M. (1999) Taxanes: an overview of the pharmacokinetics and pharmacodynamics; (Walsh, V., and Goodman, in J. Urology 54, 22-9, 1999). Considerable ancient and recent literature concern the identification of organosulfur compounds, such as (OSCs), the active components of medicial plants such as garlic, onions, Mahogany tree leaves. Cancer prevention by organosulfurates various sources are reported, from garlic and onion (Jiao, D., Smith, T. J., Yang, C. S., Pittman, B., Desai, D., Amin, S., and Chung, F. L, J Cell Biochem Suppl 27, 100-5, 1997); Reddy, B. S., Rao, C. V., Rivenson, A., and Kelloff, G., Carcinogenesis 18, 2143-7, 1993). Organosulfur compounds used for colon carcinogenesis (Wargovich, M. J. Cancer Res 53, 3493-8, 1987).

The role of sulfur compounds in anticancer chemotherapy is important due to the high affinity of sulfur-containing biomolecules like amino acids or proteins to metal-sulfur complexes, such as platinum (II). Platinum-sulfur interactions have a high impact on cellular uptake, excretion, resistance, systemic toxicity and cytotoxicity as presented by of Wang and Guo (Anti-Cancer Agents in Medicinal Chemistry 7(1):19-34, 2008). Therefore, nearly every process during platinum therapy is dependent on such interactions and it is astonishing that platinum reaches the DNA at all in the presence of sulfur. Thus, sulfur-containing molecules play significant roles in the anticancer mechanism of platinum drugs.

The characterization of coordination, ligand exchange, photophysical and redox protperties of metals ions relate with the interactions of inorganic compounds and biological molecules. Pharmaceuticals, dominated by organic drugs, now focus attention on inorganic drugs, given many activites of metals ions in biology and the development of metal-based therapeutics; inorganic drugs are likely to be transferred into the body by oxidation and ligand substitution reactions. Metal complexes are often used for treatment of a wide variety of human diseases. Inorganic medicinal chemistry began with the discovery of metal-based drugs to treat syphilis. Now, metal, metalloids elements such as platinum, titanium, bismuth, arsenic, antimony, selenium, silver, gold, vanadium, copper, manganese, germanium, iron, ruthenium, gadolinium and technetium are incorporated into many important therapeutic drugs and diagnostic imaging agents. Although twenty-six elements in the periodic table are considered essential for mammalian life, both essential and non-essential elements can be useful in drug design. [B. S. Sekhon and Leena Gandhi. Medicinal uses of inorganic compounds 1 11(49): 75-89, 2006].

Basal cell cancer (BCC) is also called non-melanoma skin cancer; ontogenically, BCCs appear to arise from pluripotential cells in the basal layer of the epidermis or follicular structures. Basal cell carcinoma accounts for more than 90 percent of all skin cancers in the United States. It is often easily detected and has an excellent record for successful treatment. Squamous cell cancirnoma (SCC), also epithelioma spinocellulare, spinalioma, spinocellular carcinoma is a non-melanoma carcinoma, a malignant tumour arising from the keratinocytes of the epidermis. It grows in a destructive way and metastasises mainly via the lymphatic system where growth can progress rapidly when extensive ulcerative necrosis occur. Keartocanthoma is a relatively common low-grade malignancy that appears to originates in the pilosebaceous glands and pathologically may closely resemble SCC. It may be treatment with excision, X-ray radiation therapy, cryosurgery with liquid nitrogen, curettage and electrodessication, intralessional therapy with methotrexate or take isotretinoin, orally.

Another important feature is that no organic materials are used to produce the compound and thus the final molecule or molecules have no carbon presence. It may behave like an organic molecule with similar activity, such as aversion for lipid or fat material, but it has no carbon presence, at least in detectable-important quantities. Further, since some of the atmosphere CO2 may dissolve in the final solution, one may check for pico or famto-mole quantities or carbon material but most would be beyond the detectable threshold of most currently available analytical equipment.

Further, the compound in solution may also behave like an amphoteric material able to operate in a wide pH range (1-8) curving or restoring the redox potential of a tissue of chemical reaction. The only limitation experienced deals with pH values exceeding 8, where the material so far renders instable and tends to precipitate.

Other teachings describing the closest subject matter provide for a number of more or less complicated aspects that fail to solve the problem in an efficient and economical way. None of these references suggest the novel features of the present invention.

### III. SUMMARY OF THE INVENTION

The antimicrobial properties of metals have been recognized for centuries and represent some of the most fundamental breakthroughs in medicinal history. Several studies stressed the role of metal ions in crucial biological processes, whereas inorganic pharmacology started to be an important field with more than twenty-five inorganic compounds with relevant applications in therapy as antibacterial, antiviral and anticancer drugs.

It has been suggested that the transfer of metal ions from ligands to cancer-associated viruses is an important mechanism for designing new anti-cancer therapies. The inverse process holds promise: coordinating a metal ion from an important biomolecule, such as zinc finger protein, recently as been used to design novel antiviral therapies, targeted against human immunodeficiency (HIV) and human papilloma virus (HPV) infections. A considerable number of metal complexes with anti-tumor activity have been reported. Sulphur-containing ligands and metal complexes of sulpher-containing amino acids have also been found to have anti-tumor activity.

There is an important relationship between metals or their complexes having antibacterial, anti-tumor, and anticancer activities. In a principal variation of the present invention, a cyclic sulfur-transition metal water soluble inorganic compound with formula S7-O4-Fe4 is obtained through inorganic chemical synthesis. The compound S7-O4-Fe4 shares biological activity with other members of the family of compounds represented with the formulation Sn-On-Xn; where S(n=5-8), O(n=3-7) and X═Fe, Cr, Ni obtained through the synthesis methodology and covered by the current invention.

Compound S7-O4-Fe4 and those previously represented by the cited general formula are generated but not limited to the procedure indicated herein. By reacting FeSO4 with Fe2O2 in equimolar amounts under high pressure and temperature using microwave energy 1.0 kjoules in an enclosed pressurized container. Metal catalysts are used to drive the reaction forward including, for example, 10.0-23.0% FeSO4, 0.01-0.08% C, 6-11% Ni, 16.1-21% Cr, 1.2-2.2% Mn, 0.032-0.045% P, 2.4-8.4 P.

Temperature (100-120 c) and pressure conditions (200-400 psi) regulate the type of compound obtained. All compounds generated exhibit particular biological and chemical activity. For the sole purpose of illustrating and supporting the present invention, only biological activity is reported herein, regarding compound s7-o4-fe4 as representative of a novel family of cyclic-sulfur-metal compounds.

The rendering compound s7-o4-fe4 or members of the family of compounds (Sn-On-Xn; where S (n=5-8), O (n=3-7) and X═Fe, Cr, Ni,), X (n=1-10) is water soluble, with a freezing point of −20 C, true boiling point 106 c.

In the present invention, a cyclic sulfur-transition metal water soluble inorganic compound with the general formula sn-on-xn; where S(n=5-8), O (n=3-7) and X═Fe, Cr, Ni), is obtained through inorganic chemical synthesis between reactants FeSO4 and H2O2. Under microwave energy of 1 kjoules and pressure of 200 psi. a compound is obtained exhibiting biological activity with a wide range of action. Said biological activity comprises anti-bacterial, anti-fungal, anti-viral, anti-neoplasic and anti-oxidant activity, apoptotic modulation, tissue repair in vitro and in vivo.

Variations of the present invention relates to the use of compound S7-O4-Fe4 and other chemical members of the group, generally represented by formulation Sn-On-Xn; where S(n=5-8), O (n=3-7) and X(X═Fe, Cr, Ni) for the preparation of compositions for cosmetic, pharmaceutical or veterinary use, intended to be administered to humans and animals for the purpose of preventing or treating disorders induced by cutaneous and mucosal pathogens.

It is reported herein that compound S7-O4-Fe4 and other chemical members of the group are capable of stabilizing and/or regulating the pathogenic flora of the cutaneous system by inhibiting the adhesion of pathogens, such as actinomycetales, streptococcus pyogenes, staphylococcus aureus, corynebacterium sp., pityrosporum ovale and candida albicans, among others.

Compound S7-O4-Fe4 and other chemical members of the group, obtained by inorganic synthesis may be chosen as active therapeutical compounds to eliminate microorganism.

Compound S7-O4-Fe4 and other chemical members of the group may be used in liquid, gel, powder and spray, suppositories, intravaginal devises and combined with other proprietary and common preparations to contribute with chemical stability, thixitropy, additional pharmacological properties, with the exception of those containing strong bases and chelating agents.

The present invention also relates to compositions for cosmetic, pharmaceutical, human or veterinary application, containing at least one compound with the formula Sn-On-Xn; where S(n=5-8), O (n=3-7) and X(n=Fe, Cr, Ni) capable of stabilizing and/or regulating cutaneous and mucosal pathogens.

The compounds reported herein under the general formula Sn-On-Xn; where S(n=5-8), O (n=3-7) and X═Fe, Cr, Ni may be used solely or as part of cosmetic and pharmaceutical preparations in the form cited herein, but not limited to liquids, creams, lotions, unguent, powder, spray, gel, dermatological solids, liposomal presentations, scalp lotions, among others.

The compounds reported herein under general formula Sn-On-Xn; where S(n=5-8), O (n=3-7) and X═Fe, Cr, Ni may be used as emollient, anti-pruritic, anti-inflamatory, antifungal, disinfectant, parasiticide, pediculicide.

The quantity of compound S7-O4-Fe4 and others reported herein under the general formula Sn-On-Xn; where S(n=5-8), O (n=3-7) and X═Fe, Cr, Ni contained in a composition may be from 0.01-50% by weight, and preferably from 9.0-20% by weight of the dry matter content.

The composition according to present invention is intended for the therapy or prophylactic treatment of healthy, sensitive and or diseased skin and/or mucous membranes which may exhibit disorders such as those induced by, but not limited to, gram positive and gram negative bacterial pathogens such as streptococcus pyogenes, staphylococcus aureus, pseudomonas, gardenella vaginalis, fungi, pityrosporum ovale, and candida albicans, viruses, such as herpes simplex I and II, herpes zoster, human papilloma virus, Venezuelan equine encephalitis, parasites and trichomonas vaginalis.

It is yet another object of this invention to provide such a compound that is inexpensive to manufacture and maintain while retaining its effectiveness.

Further objects of the invention will be brought out in the following part of the specification, wherein detailed description is for the purpose of fully disclosing the invention without placing limitations thereon.

### IV. BRIEF DESCRIPTION OF THE DRAWINGS

With the above and other related objects in view, the invention consists in the details of construction and combination of parts as will be more fully understood from the following description, when read in conjunction with the accompanying figures in which:

FIG. 1 is a diagram of a primary structure: FeS4O5 showing a sulfate group attached to sulfur-position 4.

FIG. 2 is a diagram showing a primary structure including a second sulfate, as indicated which corresponds with formula FeS5O9.

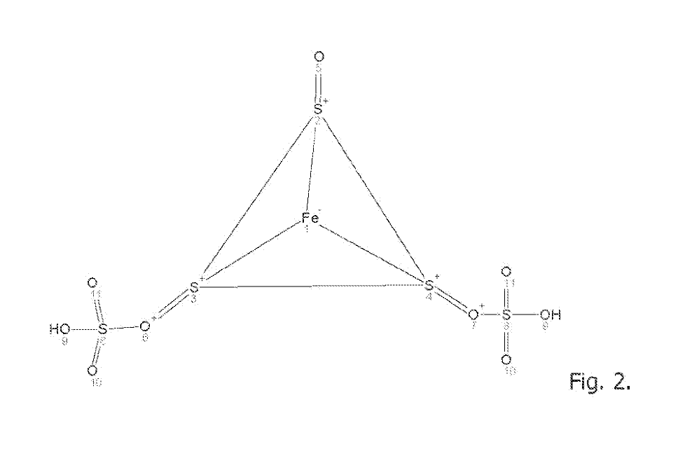

FIG. 3 is a diagram of a primary structure including a third sulfate with the structure FeS6O12.

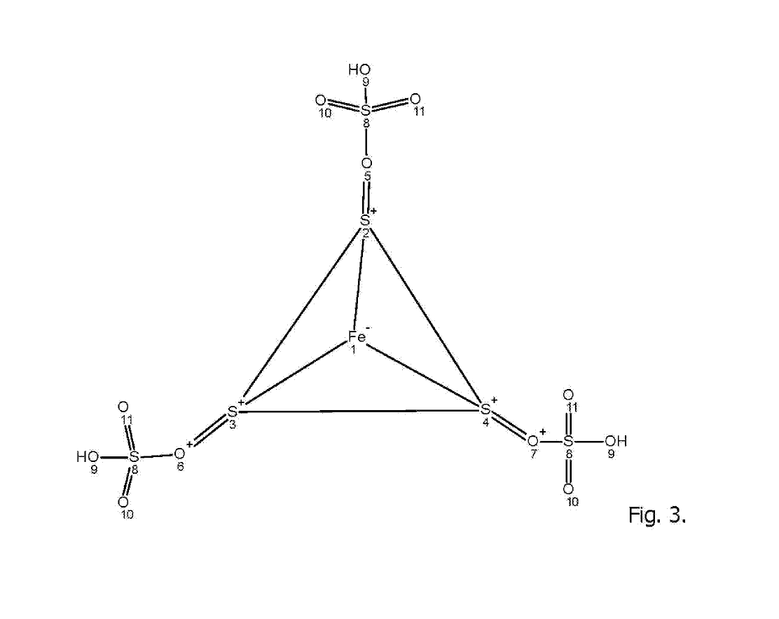

FIG. 4 is a diagram of a secondary structure comprised of several primary structures.

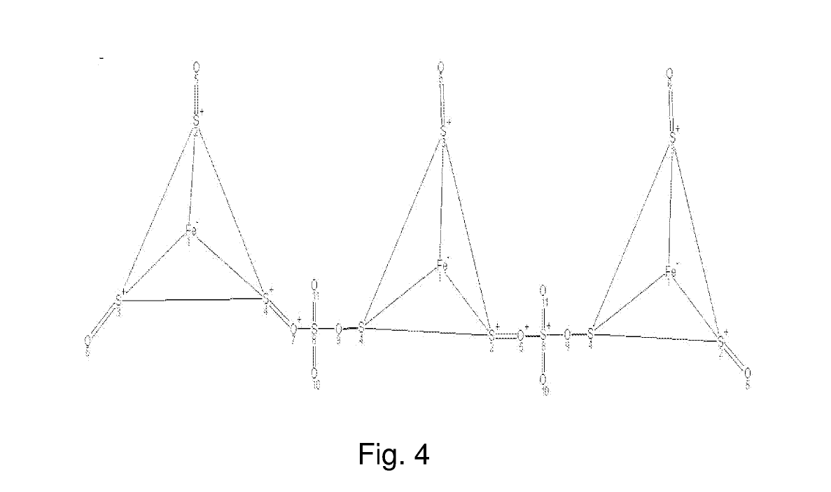

### V. DETAILED DESCRIPTION OF THE PREFERRED EMBODIMENT

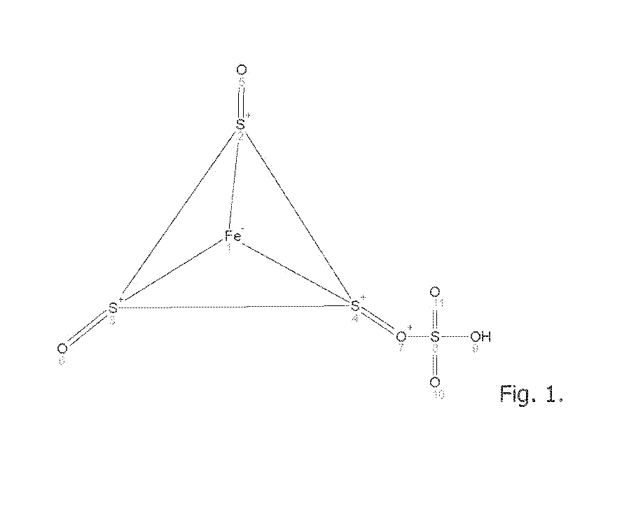

Referring now to the drawings, where the present invention is generally demonstrated by the example of the variant in FIG. 1, it can be observed that it basically includes Sn-On-X; where S(n=5-8), O (n=3-7) and X═Fe, Cr, Ni

Skin tissue covers the body of vertebrates. Where the outer layer (epidermis) is structured with dead cells, constantly sloughing and replaced from below. It helps to protect the body from infection and prevent dehydration. The lower layer (dermis) contains blood vessels, nerves, hair roots, and sweat and sebaceous glands, and is supported by a network of fibrous and elastic cells. Membranes comprise a thin layer of tissue lining all animal body cavities and canals that come into contact with the air (e.g. eyelids, breathing and digestive passages, genital tract). It contains mucus secreting goblet cells. In air passages, mucus captures dust and bacteria. The layer of cells next to the space in the tubes is lined with epithelium.

The present invention proposes the use compound S7-O4-Fe4 and others substances with the general formula Sn-On-Xn; where S(n=5-8), O (n=3-7) and X═Fe, Cr, Ni contained in a composition from 0.01-50% by weight, and preferably from 9.0-20% by weight of the dry matter content, for the preparation of compositions for cosmetic, pharmaceutical or veterinary use, intended to be administered to humans and animals for the purpose of preventing or treating disorders induced by pathogens of the skin and mucosal membranes.

Compound S7-O4-Fe4 and substances reported herein under the general formula Sn-On-Xn; where S(n=5-8), O (n=3-7) and X(n=Fe, Cr, Ni) are obtained by inorganic synthesis

Compound S7-O4-Fe4 and substances reported herein, when applied topically to the skin or mucosa, it momentarily lowers temperature by sequestering heat from surrounding media, modulating local inflammation.

Compound S7-O4-Fe4 and substances reported herein are capable of auto-generating direct electrical energy (0.5 volts) when measured with metal electrodes and, thus affecting partial charge of site, including present local microflora with partial negative charge (Leo Olitzki, The Journal of Immunology, 22: 251-256, 1932). Bacteria change their surface charge during growth cycle and are capable of inter-cell communication. This change in surface characteristics, including net charge of surface amino acids, dictates cells information interchange when forming biofilms or aggregates accompanied by local pH modulation. This is due to presence of charged macromolecules on the bacteria outer membrane.

Most bacteria survive in communities attached to each other or surfaces, including skin and mucosal membranes. Bacterial electro-kinetic cell surface properties have been shown to play a crucial role in bacterial aggregation, adhesion, interaction, formation of biofilm and stability in suspension. Bacteria are able to use a cell density dependent process, known as quorum, sensing to monitor the microbial community by producing, detecting and responding to low molecular mass signal molecules, called autoinducers (AI).

Cell-to-cell communication via quorum sensing molecules in bacteria, have been shown to be involved in bacterial interaction, including aggregation (K. Eboigbodin, 106th General Meeting of the American Society for Microbiology, May 21-25, 2006, Orlando, Fla.). This delivered charge tends to alter microbial adhesion, thus stabilize and to regulate the pathogenic bacterial flora of skin and epithelium.

Moreover, auto-generated electrical current by compound S7-O4-Fe4 and substances reported herein may affect skin voltage. A transcutaneous voltage normally exists on the skin, known as the skin battery. Where the stratum cornium is negatively charges in relation to the dermis, with a charge of 23 my (Foulds and Baker, 1983). Hairless skin surfaces display greater negative potential. It has been reported that these potentials are generated in the actively metabolizing basal region of the epidermis and an incision through the skin leads positive charge ions to the site. This is due to the sodium pump activity on the epidermal cells. Similar effects of voltage charge differences occur on healing sites.

Wound healing and therefore inflammation may be partially controlled by electrical signals affecting would healing. Compound S7-O4-Fe4 contribute to an overall local charge restoration. (J. L. Low and A. Reed, Electrotherapy explained: principles and practice, 3rd ed. Elsevier Health Sci, p. 431. 2000).

The cutaneous disorders induced by skin bacterial pathogens treatable with compound S7-O4-Fe4 and substances reported herein comprise the following etiological agents, as represented in Table 1 and corresponding primary and secondary diseases.

TABLE 1
Bacterial Pathogens

| Disease | Common agents |
|---|---|
| Primary | |
| Impetigo | Staphylococcus aureus, |
| | Streptococcus pyogenes |
| Cellulitis and erysipetas | Group A streptococci |
| Staphylococcal scalded | S aureus |
| skin syndrome | |
| Folliculitis | S aureus |
| Superficial folliculitis | |
| Staphylococcal folliculitis | S aureus |
| Gram-negative folliculitis | Klebsiella pneumeniae, Enterobacter |
| | aerogene, Proteus vulgaris |
| Propionibacterium acnes | Propionibacterium acnes |
| folliculitis | |
| Deep folliculitis | |
| Sycosis barbae | S aureus |
| Furuncles or carbunclos | S aureus |
| Pitted keratolysis | Gram-positive coryneforms |
| Erysipolied | Erysipetothrix rhusiopathiae |
| Erythrasma | Corynebacterium minutissimum |
| Trichomycosis | Corynebacterium tenuis, bacteria |
| | resembling C minutissimum, |
| | and lipophillc coryneforms |
| Secondary | |
| Intertrigo | Overgrowth of resident |
| | and transient bacteria |
| Acute inteclious eczematoid | S aureus |
| dermatilis | |
| Pseudofolliculitis of the beard | Resident flora (Gram-positive cocci) |
| Tce web infection | Fungi, corynolorm bacteria, |
| | Brevibacterium, and |
| | Gram-negative rods |
| Other diseases | |
| Mycobacterial infection | Mycobacterium tuberculosis, |
| | M marinum, M ulcerans |
| Actinomycote infection | Actinomycos lsraelil |

Primary Infections

Common or superficial impetigo (a superficial bacterial skin infection) may contain group A β-hemolytic streptococci, S aureus, or both, and controversy exists about which of these organisms is the primary pathogen. Cellulitis and erysipelas by streptococcus pyogenes is the most common agent of cellulitis, a diffuse inflammation of loose connective tissue, particularly subcutaneous tissue. Staphylococcal scalded skin syndrome (SSSS), also called Lyell's disease or toxic epidermal necrolysis, caused by phage group II staphylococci which elaborate an epidermolytic toxin.

Folliculitis can be divided into two major categories, superficial and deep. The most superficial form of skin infection is staphylococcal folliculitis. Gram-negative folliculitis occurs mainly as a superinfection in acne vulgaris patients receiving long-term, systemic antibiotic therapy. Propionibacterium acnes folliculitis has been misdiagnosed as staphylococcal folliculitis. In deep folliculitis, infection extends deeply into the follicle, and the resulting perifolliculitis causes a more marked inflammatory response than that seen in superficial folliculitis. A furuncle (boil) is a staphylococcal infection of a follicle with involvement of subcutaneous tissue. A carbuncle is a confluence of boils, a large indurated painful lesion with multiple draining sites. Erysipeloid is a benign infection that occurs most often in fishermen and meat handlers, is characterized caused by erysipelothrix rhusiopathiae.

Pitted keratolysis is a superficial infection of the plantar surface, producing a punched-out appearance produced by Gram-positive coryneform bacteria. Erythrasma is a chronic, superficial infection of the pubis, toe web, groin, axilla, and inframammary folds by Corynebacterium minutissimum. Trichomycosis involves the hair in the axillary and pubic regions and is characterized by development of nodules of varying consistency and color. Three types of coryneforms are associated with trichomycosis; one resembles C. minutissimum, one is lipolytic, and the third is C. tenuis.

Secondary Infections.

Intertrigo is a common infection seen in chubby infants or obese adults caused by overgrowth of resident or transient flora. Acute Infectious Eczematoid Dermatitis arises from a primary lesion such as a boil or a draining ear or nose, which are sources of infectious exudate. Coagulase-positive staphylococci are the organisms most frequently isolated. Pseudofolliculitis of the beard is a common disorder, occurs most often in the beard area of darker pigmented skin during and after shaving. Gram-positive resident flora are associated with this disordera as an opportunistic nonpathogenic bacteria when the host defense is impaired.

Toe web Infection, commonly referred to as athlete's foot has traditionally been regarded as strictly a fungal infection. However, fungi often cannot be recovered from the lesions throughout the disease course. Researchers now believe that the dermatophytes, the first invaders, cause skin damage that allows bacterial overgrowth, which promotes maceration and hyperkeratosis. The fungi, through the production of antibiotics, then create an environment that favors the growth of certain coryneform bacteria and Brevibacterium. Proteolytic enzymes, which are produced by some of these bacteria, may aggravate the condition.

If the feet become superhydrated, resident Gram-negative rods become the predominant flora, and the toe webs incur further damage. The fungi are then eliminated either by the action of antifungal substances of bacterial origin or by their own inability to compete for nutrients with the vigorously growing bacteria.

The cutaneous disorders induced by skin viral pathogens some treatable with compound S7-O4-Fe4 and substances reported herein comprise the following etiological agents, as represented in Table 2 and corresponding localized and generalized skin infections.

TABLE TWO

| Disease | Virus |
|---|---|
| Localized disease | |
| Herpes labiatis and herpes genitatis | Herpes simplex virus |
| Herpes zoster | Varicella zoster virus |
| Vaccinia | Vaccinia virus |
| Molluscum contagiosum | Molluscum contagiosum virus |
| Warts | Papillomavirus |
| Generalized disease | |
| Measles | Measles virus |
| Rubella | Rubella virus |
| Enteroviral exanthems and enanthems | Several enteroviruses |
| Erythema infectiosum | Parvovirus |
| Roseola | Human herpesvirus 6 |
| Hemorrhagic levers | Several logaviruses, llaviviruses, |
| | bunyaviruses, and srenaviruses |
| Smallpox (extinet) | Variola virus |

Herpes simplex virus infection is probably the most common viral skin disease. Almost every adult suffered from herpes simplex, a DNA virus and infectious agent. There are two types of herpes simplex virus. Type 1, usually associated with nongenital lesions, whereas type 2 is recovered from genital lesions. The incidence of type 1 genital infections in young patients has recently increased.

Poxviruses include closely related DNA viruses that can cause smallpox, vaccinia, and cowpox. The smallpox virus is supposedly extinct, but it can be isolated. Cowpox virus causes a cattle infection acquired by handling infected animals.

Vaccinia viruses are vaccine strains developed in the laboratory and adapted to grow in the skin of humans, rabbits, and calves. Several clinical manifestations may occur in individuals who were vaccinated against smallpox with vaccinia virus. Vaccination may produce eczema vaccinatum. Molluscum contagiosum also is caused by a poxvirus and is characterized by numerous small, pink nodules, most often on the face, genitalia, or the rectal area. Lesions also occur on the back, arms, buttocks, and inner thighs. The disease is generally harmless and self-limiting.

Papillomaviruses cause warts, where Verruca vulgaris occurs commonly on hands and fingers as single or multiple lesions. Verruca plantaris (plantar wart) is a clinical variety of verruca vulgaris that occurs on the sole of the foot. Because of the limited number of effective antiviral agents, prevention is important. Oral and intravenous acyclovir is effective for treatment of primary herpes virus infection and for recurrent genital herpes and herpes zoster in immuno-suppressed persons.

The growth of gram positive pathogens such as Staphylococcus aureus, Streptococcus pyogenes, Propionibacterium acnes and yeasts, such as Candida albicans, can cause infection and skin chemistry disequilibrium leading to disorders of the skin or mucous membranes, such as eczema, candidiasis, dermatitis, and impetigo, furunculosis, among others.

Most conventional therapies against microbial pathogens comprise but are not limited to antibiotics, chemical antibacterial agents, antiviral, parasiticidal and antifungals organic compounds. Alternatively, topical disinfectants such as ethanol, isopropyl alcohol and aldehydes may also be used.

### Fungi.

Several genera of fungi are responsible for skin diseases, including yeasts, a non-dermatophyte (Aly R, Maibach H I: Clinical Skin Microbiology. Charles C Thomas, Springfield, Ill., 1978). Fungi rarely cause disease in healthy immuno-competent hosts and infect the body through several portals of entry.

Another fungus, Malassezia furfur, is common in areas of skin rich in sebaceous glands. Both M. furfur and C. albicans are the only fungi that exist as commensals of humans and are considered part of the indigenous flora. Intertrigo is a yeast skin infections in the skin folds. Thus, a candidal infection of the mouth, known as Perleche, (or “angular cheilitis”) is a softening of the skin with deep creases around the angles of the mouth. Diaper Rash, are yeast skin infections in the diaper area.

Candidal body rash, can result from excess sweating, use of antibiotics, or skin occlusion. Candidal Vulvovaginitis, a candida infection of the vaginal tract and the external female genital area. Candidal Balanitis is a penile yeast infection. Congenital cuteneous candiadiasis, are yeast skin infections of the new borne, and results from infection of an infants skin during passage through the birth canal. Candida Paronychia is a chronic inflammation of the nail fold.

Erosiointerdigitalis blastomycetica are yeast skin infections in skin folds between fingers. Chronic mucrocutaneous candiadiasis, is an illness that results from a genetic defect that leaves those who have it with thick horny skin lesions and with nail dystrophy. Systemic Yeast Infections, are growth of candida throughout the body (Crissey J T, Lang H, parish L C: Manual of Medical Mycology, Blackwell Science, Cambridge, Mass., 1995)

Disorders of the cutaneous system may lead to yeast Skin Infections Caused antibiotics and antimycotics, after disrupting healthy skin microflora. Diabetes, given high serum glucose (sugar) levels and fat diets may predispose for candidiasis, a pathological condition of the mucous membranes; also Vaginal candidiasis, chronic eczema (homeostasis disequilibrium) on sensitive skins (premature babies, children), greasy skins (linked to hormonal dysregulations which may promote the establishment of bacteria) or to stress, damaged protective skin barrier, lowered immunity, altered bacterial flora in the gastro-intestinal tract and poor general health.

According to a preferred embodiment of the invention, compound S7-O4-Fe4 and substances reported herein can be used to treat these described skin and mucosal infections.

Compound S7-O4-Fe4 and substances reported herein may be therapeutically used at concentrations ranging from 0.01 to 50%, and most preferably from 5% to 15%.

As shown in FIG. 1, compound S7-O4-Fe4 is a cyclic metal sulfur compound synthesized by Compound S7-O4-Fe4 and those previously represented by cited general formula are generated but not limited to the procedure indicated herein. By reacting FeSO4 with Fe2O2 in equimolar amounts under high pressure and temperature using microwave energy of about 1.0 kjoules in an enclosed pressurized container. Metal catalysts are used to drive the reaction forward including 10.0-23.0% FeSO4, 0.01-0.08% C, 6-11% Ni, 16.1-21% Cr, 1.2-2.2% Mn, 0.032-0.045% P, 2.4-8.4 P.

The general formula for the compound may also have variants, which include the presence of a sulfate ion on one of the oxygen terminals. The formula HFeO6S4 (FW: 281.1087), has the composition H(0.36%) Fe(19.87%) O(34.15%) S(45.63%).

Still referring to FIG. 1, the main structure compounded by sulfur No. 4, 3, 2 is very stable and may harbor at the center the other metals represented in the general formula. Such structure is not stable without the presence of sulfate ions, given to the fact that one of the peripheral oxygen No. 5, 6, 7 is usually open to a sulphate ion tetramer such the one depicted by atoms 7,8,9,10,11. The oxygen on position will be usually protonated, and devoid of hydrogen, thus available for connection with another S-trimmer molecules, in turn generating a stable spiral or chain, contributing to its stability and reactivity. The sulfate ion acts as a ligand attaching either by one oxygen or by two oxygen as either chelate or a bridge.

The S—O bond in this sulfate is around 149 pm and shorter than expected for a single S-bond. The tetrahedral geometry predicted by valence shell electron pair repulsion (VSEPR) theory, a model in chemistry used to predict the shape of individual molecules based upon the extent of electron-pair electrostatic repulsion. The metal-sulfur structure has not been easy to define since the vibrational spectra of M-S compounds is ordinarily reported for solid structures. The spectra in the low frequency region changes substantially from solid compounds to liquid ones. Thus there is not too much information available as to the corresponding frequencies and potential continual rearrangement of the molecules in the reaction space.

The structure of our compound, we believe is involved in the capture of electromagnetic energy by the metal nucleus of the molecule and coordinated by the sulfur-metal bond vibrations, provoking a constant oxidation-reduction activated by the light spectrum, except UV range.

Orbital electronegativity, is the measure of the ability of atoms to attract electrons in the context of chemical bonds and is calculated from the partial charge distribution of atoms. Calculated at pH=7.4, it may correspond to a energy reactive compound, given the oxygen presence crown and a reactive central iron atom.

The polarizability of the DSI molecule corresponds to Mp=14.0 which comprise a vector data of a(xx)=10.98., a(yy)=10.72., a(zz)=20.29, where the electric field is generated by partial charges of the molecule, and spreads through intermolecular cavities and the solvent within the solution. The charge is measurable between metal electrodes. The induced partial charge (induced dipole) has a tendency to diminish the external electric field and this is termed polarizability. The calculation takes into account the effect of partial charges upon atomic polarizability as well as 2D and 3D geometries.

The displayed partial charge of compound DSI dictaded mainly by the oxygen radicals, which confer solvation changes, diminish surface tension and wetability, with effect on microbial water activity, survival and control.

The more stable each ionized site is, the more its vicinity is polarizable. This is why atomic polarizability is an important factor in the determination of pKa and why it is considered in our pKa calculation. The compound conformational energy corresponds with 175,29 kcal/mol, with a Van der Walls Surface area=182.07 and polar surface area: 110.1.

Most molecules contain some specific functional groups likely to loose or gain a proton under specific circumstances. Each ionization equilibrium between the protonated or deprotonated forms of the molecule can be described with a constant value called pKa. The pKa plugin calculates the pKa values of all proton gaining or loosing atoms on the basis of the partial charge distribution, as shown in Table 2.

TABLE 2
pKa data

| | |
|---|---|
| Min basica pKa | −10 |
| Max acidic pKa | 20 |
| Temp (K) | 298 |
| Micro specie pH lower limit | 0 |
| Micro specie pH upper limit | 14 |
| pH step size | 0.2 |

### Molecular Dynamics

Molecular dynamics may describe a dynamic molecular structure with a constant changing entity at various energy levels. The structure of the molecule flexibility corresponds to 183.33 kcal/mol. The Molecular Dynamics calculation propagates the positions of the atomic nuclei through a molecular mechanics force-field. The resulting trajectory can be reviewed as a molecule array or potential conformational changes given activity.

Major specie option at pH 7.4., isoelectric point with step size, pH=0.5. with complexes between electron donors and acceptors have been researched extensively, and are defined as strong or weak ionic depending on the charge transfer. Organic and inorganic molecules have been used as electron donors (C. K. Prout, R. J. O. Williams and J. D. Wright, J. Chem Soc A:747, 1966)

The isoelectric point (pI) is the pH at which a molecule carries no net electrical charge. The isoelectric point plugin calculation considers all macro ionization states of an ionizable molecule across the pH range. The net charge at given pH is calculated from the weighted sum of the macro distribution.

Proteins have minimal solubility at their isoelectric point. The mobility of molecule is minimal at pI and so an important parameter for electroanalytical separation techniques as shown in Table 3.

TABLE 3
Log data (weighted)

| Variables | Log P | LogD |
|---|---|---|
| VG | 1.0 | 1.0 |
| KLOP | 1.0 | 1.0 |
| PHYS | 1.0 | 1.0 |
| Cl⁻ concentration | 0.1 | 0.1 |
| (mol/dm³) | | |
| Na⁺K⁺ concentration | 0.1 | 0.1 |
| (mol/dm³) | | |
| pH lower limit | — | 0 |
| pH upper limit | — | 14 |
| pH step size | — | 0.5 |
| Ref pH values = 1 | — | 1.5 |
| Ref pH values = 2 | — | 5.0 |
| Ref pH values = 3 | — | 6.5 |
| Ref pH values = 4 | | 7.4 |

The logP plugin calculates the octanol/water partition coefficient, which is used in QSAR analysis and rational drug design as a measure of molecular hydrophobicity. The calculation method is based on the publication of Viswanadhan at al. The logP value of a molecule is composed of the increment values of its atoms. Though, logP is generally calculated for the neutral molecule forms only. While for the value of LogD, compounds having ionizable groups exist in solution as a mixture of different ionic forms. The ionization of those groups and so the ratio of the ionic forms depends on the pH. Since logP describes the hydrophobicity of one form only, the apparent logP value can be different. The octanol-water distribution coefficient, logD represents the compounds at any pH value.

Molecular representation of compound FeO6S2 which may rearrange into compound S7-O4-Fe4 as repetitive unit and conforming a three dimensional structured molecules is demonstrated in Tables 4 and 5.

TABLE 4
Some characteristics of Compound FeO6S2

| Abrev. | Characteristic | Values |
|---|---|---|
| MF | Molecular Formula | FeO6S2 |
| FW | Formlual Weight | 215.970 g/mol |
| Mmi | Monoisotropic | 215.847 Da |
| | Mass | |
| Mn | Nominal Mass | 216 Da |
| Mav | Average Mass | 215.970 Da |
| [M + H]− | | 216.854 Da |

TABLE 5
Composition and Molecular Weight of Compound FeO6S2

| | | Molar Mass (MM) | Subtotal Mass | Subtotal Mass |
|---|---|---|---|---|
| # | Atom | (g/mol) | (%) | (g/mol) |
| 1 | Fe | 55.85 | 25.86 | 55.85 |
| 6 | O | 16.00 | 44.45 | 96.00 |
| 2 | S | 32.07 | 29.69 | 64.13 |

| | |
|---|---|
| Total Molecular Weight: | 215.97 |

### Huckle Analysis and Localization Energies

FIG. 5 shows an example of the localization energies L(+) and L(−) for electrophilic and nucleophilic attack at an cyclic center were calculated by the Huckel method. The smaller L(+) or L(−) means more reactive atomic location. Order of atoms in E(+) or in Nu(−) attack are adjusted according to their localization energies. The total pi energy for compound FeO6S2 is 27.07.

### Charge Density

FIG. 6 shows an example of partial charge distribution determines many physico-chemical properties of a molecule, such as ionization constants, reactivity and pharmacophore pattern.

The partial charge value of each atom calculated, where total charge is calculated from sigma and pi charge components. When the compound combines, an electron transfer occurs that mainly depends on the electronegativity difference between them. The atom, initially higher in the electronegativity, attracts electrons. It must acquire partial negative charge and therefore decreases. The other atom acquires partial positive charge and its negativity becomes equal. It has been shown that electronegativity actually corresponds to the electronic chemical potential which at the end is a thermodynamic principle of chemical potential equalization.

Compound FeO6S2 actually depolarizes the surface charge of surface hydrophilic or hydrophobic amino acids present on the microbial cell wall or partial capsule as well as in virus active sites required for cell adsorption and infectivity.

Molar Refractivity is about 19.98

Molar refractivity is strongly related to the volume of the molecules and to London dispersive forces with important effect in drug-receptor interaction. Compound FeO6S2 displays a polar surface=110.10. It is formed by polar atoms of a molecule. It is a descriptor that shows good correlation with passive molecular transport through membranes, and so allows estimation of transport properties of drugs.

In a variation, the unit fragment of compound FeO6S2 may conform into a multiple tridimensional stereo isomers, such as S7O6Fe.

A water solution of inorganic compound FeO6S2 has biological activity within an operative pH range of 6.0-8.5, with optimal activity near physiological pH (7.40).

A water solution of inorganic compound FeO6S2 is non functional at extreme pH<6.0 and >8.5; and in presence of strong bases (NaOH, KOH) or strong inorganic acids (H2SO4, HNO3, HF, H3PO4), also with solutions of barium sulfate, at pH below 6 or above 9.

There is no upper limit for the number of ionizable atoms in the molecule and so the pKa plugin can also be used for proteins. Acidic and basic molecules are ionized in aqueous solution. Acidic or basic character is assigned to the molecule according to Brönsted's rule. The ratio of the ionized and neutral forms depends on the pH, the temperature and the ion activity of the bulk phase. The ionization constant Ka is obtained from the activity ratio of conjugated base and conjugated acid multiplied with proton activity.

Molecular Dynamics

The iron molecule it is not actually imbedded in the chemical structure or directly part of it as we thought but an ionic ligand, contributing with structural molecular stability in the presence of water. The rest of the molecule remains the same.

Molecular Models

The primary compound, presented as a monomer may co-exist temporarily as a single compound but to rearrange into two or more forms until stability is reached. See molecular models. The proposed metal polysulfide structure corresponds to two general formulas H2FeO[HmO2n+2S2n−1] and H2FeO[HmO2n+3S2n−1]; where, m may be 3-4; n may be 1-25 and p may be 4-25; while the optimal composition for electrochemical activity appears to be H2FeO[H4O15S14]. Thus, the monomer or singlet with a chemical formula of H2FeO.[H3O4S3].

The second degree-dimer (B1) chemical formula is H2FeO.[H4O6S6] and structure. Also, the S═S bond may include an oxygen atom to yield a slightly different but more reactive compound (B2), H2FeO[HmO2n+3S2n−1].

Any of the compounds subject to synthesis using the present technology may be represented with the general formula (XnSnOn), with a range of X(1-8) S(4-32) O(4-41); where X can be any metal in the periodic table with valance 3 or 6 or both (at least), such as Fe, Ni, Cr, Al, Bo, Ti, V, Co, Ru, etc); n, the number of atoms. S, is sulfur and n, the number of atoms. O, oxygen, where n, is the number of atoms.

The present invention is based on the surprising discovery that compound S7-O4-Fe4 and other chemical members of the group represented with the general formula Sn-On-Xn; where S(n=5-8), O (n=3-7) and X(n=Fe, Cr, Ni) exerts cytotoxic effects on a number of cancer cell types. It has also been discovered that the compound subject of this report may exerts its toxic effects through distinct modes of action, as compared to other anti-cancer drugs.

Compound S7-O4-Fe4 and other chemical members of the group, represented by the general formula Sn-On-Xn; where S(n=5-8), O (n=3-7) and X(n=Fe, Cr or Ni) are inorganic in nature and water soluble, display a wide anti-microbial activity, are non-infectious, non-corrosive, non-mutagenic, non-ionizing and considered general stable over time.

Compound S7-O4-Fe4 and other chemical members of the group, represented by the general formula Sn-On-Xn; where S(n=5-8), O (n=3-7) and X(n=Fe, Cr or Ni) in liquid form may be stored at room temperature and pressure in light-unprotected glass.

### Synthesis

This invention relates to the synthesis of polysulfide FemSnOp; where m may be 1-8; n may be 4-32 and p may be 4-32 and optimal composition for electrochemical activity is FemSnOp where m may be 1-8; n may be 4-8 and p may be 4-8, in an electrochemical cell having heavy metal electrodes and containing hydrosoluble ionic sulfur compounds under said pressure, temperature and voltage conditions. As per described in patent application Ser. No. 06/853,098 no polysulfide electrodes are used in the proposed process, in addition of using sulfur containing aqueous electrolytes and generating a hydrosoluble polysulfide. The polysulfide compound mentioned and generated with the methodology presented herein exhibits electrical conductivity, low vapor pressure and dissolution rate.

Commonly assigned U.S. Pat. No. 4,481,267 discloses a novel class of metal polysulfide materials utilized as cathodes in non-aqueous electrochemical cells. These metal polysulfides are characterized by having an atomic ratio of sulfur to transition metal of greater than 3.5 to 1. These materials come close to the theoretical capacity of sulfur, having theoretical capacities slightly greater than 1.0 A-Hr/g. In the invention presented herein the metal polysulfide is a hydrosoluble ionic compound and unrelated to the electrodes used. The hydrosoluble ionic polysulfide presented herein is generated in liquid form and different from heavy metal polysulfides prepared by precipitation from mixing an aqueous solution of the metal chloride salt with an aqueous polysulfide solution, such as presented in U.S. application No. 769,036 discloses a method of preparing heavy metal polysulfide using ammonium polysulfide; U.S. Pat. No. 4,481,267 are not heated to have a sulfur to metal ratio from 3.5/1 to as high as 5/1; U.S. Pat. No. 4,481,267 it has been found that there is less degradation on storage if the heavy metal polysulfides are first heated to a constant weight value before being made into cathodes. Heating under vacuum to a constant weight removes loosely bound sulfur and results in polysulfides having a sulfur content from between 3.5/1 to 4.5/1. A mixture of heavy metal polysulfides may be generated in the resulting electrolyte after reaction X(1)a+n X(2)b+m (Sx)c2 wherein X(1) and X(2) are different heavy metal atoms, n and m are integers representing the valence states of X(1) and X(2) respectively, a and b are non zero integers representing the stoichiometry of X(1) and X(2) respectively in the polysulfide, S is sulfur, na+mb=2c and x is greater than 4.5. When mixture of soluble sulfur compound are present in the electrolyte reaction solution and conditioned as previously indicated, the generated polysulphide exhibits a higher electrochemical capacity.

The present invention pertains the generation of a novel metal polysulfide compound in an electrochemical cell containing an aqueous solution with sulfate ions in the presence of heavy metal plates. Specifically, when an aqueous equimolar solution of sulfate ions where the metal polysulfide FemSnOp is generated, where m may be 1-8; n may be 4-32 and p may be 4-32 and optimal composition for electrochemical activity is FemSnOp where m may be 1-8; n may be 4-8 and p may be 4-8, in an electrochemical cell having heavy metal electrodes and containing hydrosoluble ionic sulfur compounds under said pressure of 300 PSIG, temperature 60 C and DC voltage conditions 100 amperes. A precipitate is generated consisting of oxides and hydroxides of participating metals which are removed by filtration. It is an advantage of the present invention that mixed heavy metal polysulfides can be prepared after using anode and cathode active materials in the electrochemical cells, alloys of different heavy metals selected from a group consisting of copper, iron, chromium, nickel and cobalt, and where the source of sulfur is contained within the electrolyte; and wherein S is sulfur and y is greater than or equal to 4.5. The electrolyte used in the cells is aqueous. It is an additional advantage of this invention is that the heavy metal polysulfides exhibit properties different to corresponding single metal polysulfides. For example, Fe 3 S 8 is very air sensitive and decomposes readily while FemSnOp is stable and capable of chemical-electrical properties such as conductivity.

It is another object of the present invention to provide polysulfide materials that are cheaper to produce with similar and novel properties than reported polysulfides. Advantages and objects of present invention will become clear in light of the following examples. Examples are illustrative in nature and a variety of heavy metal polysulfides can be prepared with heavy metal electrodes with the compositions mentioned herein. Accordingly, the details described herein in such examples are not limited to the variables selected. Values presented are percent by weight.

### Example 1

An aqueous solution comprise equimolar amounts of sodium sulfate, sodium chloride and calcium carbonate, magnesium chloride are reacted in an electrolytic cell in presence of an electrical AC field of 6-120 amp for 15 minutes under pressure at 150 PSIG between two heavy metal alloy electrodes containing iron, stainless steel, carbon steel, grey iron, and having trace content of Ni, Cr, Mo . . . with the general formula X(1)a+n X(2)b+m (Sx)c2 wherein X(1) and X(2) are different heavy metal atoms, n and m are integers representing the valence states of X(1) and X(2) respectively, a and b are non zero integers representing the stoichiometry of X(1) and X(2) respectively in the polysulfide, S is sulfur, na+mb=2c and x is greater than 4.5. The reacting aqueous electrolytic solution may contain ionic metals and rare earth elements in trace amounts (>0.5) to enhance the reaction to yields the formation of polysulfide FemSnOp; where m may be 1-8; n may be 4-32 and p may be 4-32 and optimal composition for electrochemical activity is FemSnOp where m may be 1-8; n may be 4-8 and p may be 4-8. Also, a dark precipitate generated containing oxidized metals, where the dark precipitate is removed, dried and used in other applications not cited herein.

### Example 2

An aqueous solution comprise equimolar amounts of sodium sulfate, iron sulfate, hydrogen sulfide, sodium chloride and calcium carbonate are reacted in an electrolytic cell in presence of an electrical AC field of 6-120 amp for 15 minutes under pressure at 150 PSIG between two heavy metal alloy electrodes containing iron, stainless steel, carbon steel, grey iron, and having trace content of Ni, Cr, Mo . . . with the general formula X(1)a+n X(2)b+m (Sx)c2 wherein X(1) and X(2) are different heavy metal atoms, n and m are integers representing the valence states of X(1) and X(2) respectively, a and b are non zero integers representing the stoichiometry of X(1) and X(2) respectively in the polysulfide, S is sulfur, na+mb=2c and x is greater than 4.5. The reacting aqueous electrolytic solution may contain ionic metals and rare earth elements in trace amounts (>0.5) to enhance the reaction to yields the formation of polysulfide FemSnOp; where m may be 1-8; n may be 4-32 and p may be 4-32 and optimal composition for electrochemical activity is FemSnOp where m may be 1-8; n may be 4-8 and p may be 4-8. Also, a dark precipitate generated containing oxidized metals, where the dark precipitate is removed, dried and used in other applications not cited herein.

### Example 3

An aqueous solution comprise equimolar amounts of calcium sulfate, sodium chloride, iron sulfate and calcium carbonate are reacted in an electrolytic cell in presence of an electrical AC field of 6-120 amp for 15 minutes under pressure at 150 PSIG between two heavy metal alloy electrodes containing iron, stainless steel, carbon steel, grey iron, and having trace content of Ni, Cr, Mo . . . with the general formula X(1)a+n X(2)b+m (Sx)c2 wherein X(1) and X(2) are different heavy metal atoms, n and m are integers representing the valence states of X(1) and X(2) respectively, a and b are non zero integers representing the stoichiometry of X(1) and X(2) respectively in the polysulfide, S is sulfur, na+mb=2c and x is greater than 4.5. The reacting aqueous electrolytic solution may contain ionic metals and rare earth elements in trace amounts (>0.5) to enhance the reaction to yields the formation of polysulfide FemSnOp; where m may be 1-8; n may be 4-32 and p may be 4-32 and optimal composition for electrochemical activity is FemSnOp where m may be 1-8; n may be 4-8 and p may be 4-8. Also, a dark precipitate generated containing oxidized metals, where the dark precipitate is removed, dried and used in other applications not cited herein.

### Example 4

An aqueous solution comprise equimolar amounts of sulfides of alkali ions (Li+,Na+,K+,Rb+,Cs+,Fr+), iron, alkali earth metals (Be2+,Mg2+,Ca2+,Sr2+,Ba2+,Ra2+), and H+ (aq), NH4+, and calcium carbonate are reacted in an electrolytic cell in presence of an electrical AC field of 6-120 amp for 15 minutes under pressure at 150 PSIG between two heavy metal alloy electrodes containing iron, stainless steel, carbon steel, grey iron, and having trace content of Ni, Cr, Mo . . . with the general formula X(1)a+n X(2)b+m (Sx)c2 wherein X(1) and X(2) are different heavy metal atoms, n and m are integers representing the valence states of X(1) and X(2) respectively, a and b are non zero integers representing the stoichiometry of X(1) and X(2) respectively in the polysulfide, S is sulfur, na+mb=2c and x is greater than 4.5. The reacting aqueous electrolytic solution may contain ionic metals and rare earth elements in trace amounts (>0.5) to enhance the reaction to yields the formation of polysulfide FemSnOp; where m may be 1-8; n may be 4-32 and p may be 4-32 and optimal composition for electrochemical activity is FemSnOp where m may be 1-8; n may be 4-8 and p may be 4-8. Also, a dark precipitate generated containing oxidized metals, where the dark precipitate is removed, dried and used in other applications not cited herein.

### Comparative Example A

Electrolytic cell may be constructed in epoxy resin coated stainless steel pressure resistant reaction vessel of rectangular or cylindrical shape complemented with pair electrodes, where several pair electrodes may be arrange in series or parallel. A typical lab size cell my contain rectangular electrodes with dimensions of 20×20 cm of metal material, having 2 mm thickness. The cell jacketed for water vapor circulation and heating to condition the reaction temperature. The cell assisted with a water pump for electrolytic cell fluid recirculation. Electrical AC current 6-120 amp applied to the electrodes regulated by a cutoff timer that also regulates water pump operation, usually set for 15 minute. Head space within the cell corresponds to about 20% available space where a vacuum line is connected to lower the solution boiling point; where the operation temperature is 60 C. The cell operation proceeds until electrolytic aqueous liquid no longer supports conductance; indicative of reaction completion. In order to take full advantage of the high energy densities of the polysulfide materials generated through the present invention it is preferred the use of an aqueous cells having heavy metal alloy electrodes electrodes containing iron, stainless steel, carbon steel, grey iron, and having trace content of Ni, Cr, Mo. Although the heavy metal polysulfide generated and represented by the general formula FemSnOp given in the examples were limited to its generation through three different heavy metals type electrodes it would be possible to prepare other polysulfides or mixtures therein when using other electrolytic mixtures containing other concentrations or sulfur sources and heavy metal electrodes with different composition.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. Therapeutic use of the chemical compound of the group represented by the formulation Sn-On-Xn; where S(n=5-8), O (n=3-7), X═Fe, Cr or Ni and X(n=1-25) obtained through inorganic chemical synthesis used in the preparation of a composition for cosmetic and pharmaceutical use, intended to be externally topically administered to humans and animals for the purpose of preventing or treating disorders of the skin and mucosal membranes induced by indigenous and transient microflora.

2. Therapeutic use of the chemical compound of the group represented in claim 1 more particularly and further characterized by the formula S7-O4-Fe4.

3. Therapeutic use of compound Sn-On-X; where S(n=5-8), O (n=3-7) and X═Fe, Cr or Ni for use in stabilizing and/or regulating the presence of pathogenic flora of the cutaneous and mucosal tissues, including bodily orifices, by inhibiting but not limited to, adhesion of pathogens through changing cell membrane polarity, modification of target cell surface receptor conformation, modulating cellular apoptosis and oxidation or ion concentration modulation.

4. Therapeutic use of said compound as disclosed in claim 3 further characterized in that said pathogen groups are comprised of any singly or combination of virus, bacteria, fungi and parasites

5. Therapeutic use of said compound as disclosed in claim 4 further characterized in that said pathogen groups may be comprised of any one or combination of Herpes simplex I, Herpes simplex II, Human Papilloma Virus, Coxackievirus, Actinobacillus actinomycetemcomitans, Pseudomonas sp., Porphyromonas gingivalis., Escherichia, sp., Enterococcus, Staphyloccocus sp. Streptococcus sp., Escherichia coli, Streptococcus pyogenes, Candida sp., Pityrosporum ovale, and M. furfur.

6. Therapeutic use of said compound as disclosed in claim 1 further characterized in that used in a concentration of 0.01-50% w/v.

7. Therapeutic use of said compound as disclosed in claim 2 further characterized in that used in a concentration of 0.01-50% w/v.

8. Therapeutic use of said compound as disclosed in claim 4 further characterized in that used in a concentration of 0.01-50% w/v.

9. Therapeutic use of said composition according to claim 1, in which disorders of cutaneous, dermo-epithelial and mucosal systems, intradermal and subcutaneous areas including bodily orifices may be subjected to infection and disease, where treatment of resulting lesions comprise but are not limited to superinfected atopic dermatitis, impetiginized eczema, superinfected inflammatory acne, polydermatites such as impetigo, superficial folliculites, seborrhoeic dermatites, Pityriasis versicolor, dermatomycosis, candidiasis, ulcers, wounds, burns and those linked to therapeutic treatments with antivirals, antibiotics or antimycotics, conjunctivitis, folliculitis, acne, gingivitis, fungal, parasitosis, stomatitis, condylomatosis, uretitis, balanitis, vaginitis, bacteriosis and endometriosis on skin and mucosa of body, rectum, genitalia, vagina, uterus, urethra, mouth, nose, throat, eyes, ear and auditory canal.

10. Therapeutic use of said composition according to claim 2, in which disorders of cutaneous, dermo-epithelial and mucosal systems, intradermal and subcutaneous areas including bodily orifices may be subjected to infection and disease, where treatment of resulting lesions comprise but are not limited to superinfected atopic dermatitis, impetiginized eczema, superinfected inflammatory acne, polydermatites such as impetigo, superficial folliculites, seborrhoeic dermatites, Pityriasis versicolor, dermatomycosis, candidiasis, ulcers, wounds, burns and those linked to therapeutic treatments with antivirals, antibiotics or antimycotics, conjunctivitis, folliculitis, acne, gingivitis, fungal, parasitosis, stomatitis, condylomatosis, uretitis, balanitis, vaginitis, bacteriosis and endometriosis on skin and mucosa of body, rectum, genitalia, vagina, uterus, urethra, mouth, nose, throat, eyes, ear and auditory canal.

11. Therapeutic use of said composition according to claim 1, for human and veterinary use, for the treatment or prevention of infection and colonization by gram positive bacteria and fungal infections.

12. Therapeutic use of said composition according to claim 2, for human and veterinary use, for the treatment or prevention of infection and colonization by gram positive bacteria and fungal infections.

13. An aqueous electrochemical cell comprising a metal anode, cathode and an electrolyte, all enclosed in pressurized chamber, 150 PSIG; The electrodes comprise made of various metal alloys and heavy metal alloys, including stainless steel, carbon steel, grey iron, and having content of Ni, Fe, Cr, Fe with the general formula X(1)a+n X(2)b+m (Sx)c2 wherein X(1) and X(2) are different heavy metal atoms, n and m are integers representing the valence states of X(1) and X(2) respectively, a and b are non zero integers representing the stoichiometry of X(1) and X(2) respectively in the polysulfide, S is sulfur, na+mb=2c and x is greater than 4.5.

14. The electrochemical cell of claim 13 wherein said heavy metals (X) include copper, iron, chromium, titanium, nickel and cobalt.

15. The electrochemical cell of claim 13 wherein the anode and cathode may contain rare earth metals in trace amounts.

16. The electrochemical cell of claim 13 wherein said electrolyte is a Newtonian aqueous fluid and mixture of sodium sulfate 2-14%, iron sulfate 7-10%, calcium sulfate 0.5-5%, sodium chloride 1-10%, magnesium chloride 0.5-7% and calcium carbonate 1-23%.

17. The electrochemical cell of claim 13 wherein said electrolyte is aqueous liquid.

18. The electrochemical cell of claim 13 is for aqueous solutions comprising a heavy metal cathode and anode having an empirical stoichiometric formula X(1) X(2)Sy wherein X(1) and X(2) are alloys of different heavy metals selected from a group consisting of copper, iron, chromium, nickel and cobalt, and where the source of sulfur is contained within the electrolyte; and wherein S is sulfur and y is greater than or equal to 4.5; The obtained key polysulphide compound once synthesized remains soluble in solution while metal oxides and hydroxides of present soluble metals are generated and precipitate as insoluble and removable material.

19. The reaction cell operated at 60 C under 200 PSIG at 6-120 AC Amps under vacuum during 15 minutes to obtain said material.
